(19) United States
(12) Patent Application Publication
(10) Pub. No.: US 2013/0138090 A1
(43) Pub. Date: May 30, 2013

(54) RELEASE DEVICE FOR RELEASING A MEDICAL IMPLANT FROM A CATHETER AND CATHETER COMPRISING A RELEASE DEVICE

(71) Applicant: BIOTRONIK AG, Buelach (CH)

(72) Inventor: Amir Fargahi, Buelach (CH)

(73) Assignee: BIOTRONIK AG, Buelach (CH)

(21) Appl. No.: 13/681,232

(22) Filed: Nov. 19, 2012

Related U.S. Application Data
(60) Provisional application No. 61/563,554, filed on Nov. 24, 2011.

(51) Int. Cl. A61B 17/00

(52) U.S. Cl. CPC A61B 17/00
USPC 606/1; 29/428

(57) ABSTRACT

A release device (100, 100a) or an insertion device (110) including the release device for releasing a medical implant (105) from an insertion device (110), in the case of which the implant (105) can be released by way of a relative motion between a first and a second insertion element (52, 54), comprising a body (10) having a proximal end (12) which faces a user during use, and a distal end (14) which is remote from the user during use, wherein an actuator (16) is provided between the proximal and the distal end (12, 14), wherein the actuator (16) can be driven hydraulically to generate a targeted relative motion between the first and the second insertion element (52, 54) of the insertion device (110).

### CROSS REFERENCE TO RELATED APPLICATIONS

This application claims benefit of priority to U.S. provisional patent application Ser. No. 61/563,554, filed Nov. 24, 2011; the content of which is herein incorporated by reference in its entirety.

### TECHNICAL FIELD

The invention relates to a release device for releasing a medical implant from a catheter and a catheter comprising a release device for releasing a medical implant for implantation in an animal body and/or human body.

### BACKGROUND

Implants are used often in medical applications for implantation in an animal body and/or human body permanently or at least for an extended period of time to perform replacement functions. Examples would be e.g. cardiac pacemakers, brain pacemakers for Parkinson's patients, cardiac implants, cochlear implants, retinal implants, dental implants, joint replacement implants, vascular prostheses or stents.

Implants are connected to catheters for insertion into the body and it must be possible to place them precisely and release them in a defined manner at the application site. To this end, it is known, for example, to release the implant by way of a sliding motion.

### SUMMARY

The problem addressed by the invention is that of providing a release device by way of which an implant can be released in a highly precise and targeted manner.

A further problem addressed is that of providing a related insertion device.

A further problem addressed is that of providing a method for manufacturing such a release device.

Yet another problem addressed is that of providing a method for operating a related release device.

A release device for releasing a medical implant from an insertion device is provided, in the case of which the implant can be released by way of a relative motion between a first and a second insertion element. The release device comprises a body having a proximal end which faces a user during use and a distal end which is remote from the user during use, wherein an actuator is provided between the proximal end and the distal end, and wherein the actuator can be driven hydraulically to generate a targeted relative motion between the first and the second insertion element of the insertion device.

By way of the embodiment according to the invention, a release device can be provided, in the case of which large forces can be easily generated. It is thereby possible to minimize a force to be applied by the user and/or physician in a user-friendly manner. The user and/or physician can concentrate on correct positioning of the implant. The release of the implant becomes more precise and rapid as a result. All of this results in a high success rate for implantation. In this context, “provided” is intended to mean, in particular, specially equipped and/or designed.

It is furthermore provided that the body comprises a valve, at least on the distal end. The valve is preferably a valve which can be operated from the outside. However, it can also be a media-controlled valve that can be operated in one direction, e.g. for ventilation which takes place due to a pressure increase. In the structurally simplest case, the valve is in the form of a closeable luer lock. Standard components and/or connection dimensions can therefore be used, to advantage. Advantageously, a valve is provided on the proximal end and on the distal end, thereby enabling the release device to be used for a plurality of operating modes in a component-sparing manner.

Furthermore, it is advantageous when a hydraulic fluid for moving the actuator can be introduced into the body by way of the valve. This design enables the force application to be easily regulated and controlled. Due to the low compressibility of hydraulic fluid, a particularly stable movement of the insertion element can be achieved. The hydraulic fluid can be introduced, preferably, by way of a syringe, thereby enabling a conventional means to be used, the mode of operation of which is known. An orientation phase, which is time-consuming, is therefore eliminated. The hydraulic fluid is preferably water. The water or the hydraulic fluid reduces the friction in the body, thereby advantageously minimizing the internal friction in the release device.

In a further embodiment of the invention, a speed of the relative motion between the first and the second insertion element of the insertion device can be specified by way of a hydraulic pressure. It is thereby possible to achieve a rapid, uniform and/or steplessly variable or controllable speed. A stepped adjustment would also be feasible, in principle. The use of pressure can also enable the implant to be positioned in a highly precise manner, instead of sliding and retracting the insertion elements as in the prior art. A pressure to be applied is dependent upon a length and/or a diameter of the body and/or the dimensions or effective surfaces of the actuator. A person skilled in the art will make the selection himself according to his expertise.

It is furthermore provided that at least two pressure ranges are provided, wherein a first, lower pressure range is provided for a slow relative motion, and a second, higher pressure range is provided for a faster relative motion. This enables easy implementation of at least two different speeds of the release device. In addition, advantageously, it is easy to switch from a rapid release to a slow release, and vice versa. This results in uncomplicated, simple and rapid speed regulation during release of the implant. If a pressure of 2 bar is applied to the actuator, for example, a slow relative motion or release is induced. However, if a pressure of 6 bar is applied, the relative motion and, therefore, the release is faster.

Furthermore, it can be advantageous when the actuator comprises a passage for the inner insertion element. This results in a compact configuration which stabilizes and protects the insertion element extending therethrough. If the insertion device is a catheter, the applicable insertion element can be an internal shaft of the catheter.

Efficient force transmission having sufficient contact surface can be advantageously achieved when the actuator comprises a plunger rod and a plunger. It is furthermore provided that the plunger is disposed at a proximal end of the plunger rod, whereby, during the motion, the plunger rod can move into an insertion device adjoining the body distally in a space-saving manner. The result is an advantageously simple and compact design of the release device.

According to a further aspect of the invention, it is provided that the body comprises a hydraulic cylinder for the plunger, thereby resulting in a stable design. Preferably, the body forms the hydraulic cylinder. As a result, components, assembly effort and costs can be advantageously spared. In addition, a low weight of the insertion device is achieved.

In a preferred development, the plunger rod is designed as one piece with the outer insertion element, in particular the outer shaft in the case of a catheter, thereby enabling the relative motion to be transmitted particularly reliably and exactly. Furthermore, components can be omitted as a result, and the weight can be reduced further. Advantageously, the plunger is designed as one piece with the outer insertion element. This enables force to be transmitted efficiently and directly to the insertion device and results in a component-reducing and compact design of the insertion device.

According to an advantageous embodiment, the release device comprises at least one seal, thereby advantageously preventing the loss of hydraulic fluid, which stabilizes the process. The seal can be in the form of any element considered reasonable by a person skilled in the art, such as an O ring. The seal is preferably made of a material which is resistant to the hydraulic fluid and has good antifrictional properties, in particular on the insertion element. It is furthermore provided that a seal is disposed between at least one insertion element and the body. As a result, the body can be advantageously sealed with respect to surroundings of the body. In the case of a catheter, the insertion element is preferably the outer shaft. It is furthermore advantageous when a seal is disposed between an insertion element and the actuator, thereby preventing hydraulic fluid from entering the space between the insertion elements. In the case of a catheter, the insertion element is preferably the inner shaft. According to an advantageous embodiment, a lumen of the inner insertion element can comprise a ventilation valve.

In a further embodiment of the invention, the body is in the form of a housing, thereby enabling the body and the release device to be very stable. The housing can form a handle of the release device or the insertion device in particular. This permits the release device to be operated easily and in a controlled manner. The housing is advantageously transparent in at least one region, thereby enabling particularly easy monitoring of the progress of the movement of the actuator and, therefore, the insertion element. Preferably, the entire housing is transparent.

In a preferred embodiment, the speed of the relative motion can be monitored visually. This can take place in a particularly simple manner by monitoring the movement of the colored (e.g. black) plunger in the transparent housing or cylinder. For this purpose, the housing or the cylinder preferably has a scale, such as a mL, scale.

Furthermore it is provided that a speed of the relative motion can be monitored and/or regulated by way of a syringe manometer (inflation syringe manometer). This enables the load to be displayed easily. A further control means can thereby also be provided, which is independent of an observation of the actuator movement in particular. This observation can also be carried out by a person other than the user of the release device and is independent of an unobstructed view of the body or the housing.

Furthermore, an insertion device for inserting a medical implant is provided, which can be released by way of a relative motion between a first and a second insertion element. The insertion device comprises a release device for releasing the medical implant, which comprises a body having a proximal end which faces a user during use and a distal end which is remote from the user during use, wherein an actuator is provided between the proximal end and the distal end, and wherein the actuator can be driven hydraulically to generate a targeted relative motion between the first and the second insertion element of the insertion device.

By way of the embodiment according to the invention, an insertion device can be provided, in the case of which large forces can be easily generated independently of the force to be applied by the user or the physician. In addition, it is easy to use and has an optimized design. The release of the implant becomes more precise and rapid as a result. All of this results in a high success rate for implantation. The insertion device can be a catheter, which is favorable. Particularly advantageously, the insertion device can be used to install and release a prothesis, a heart valve or a stent.

A method for manufacturing a release device comprising an implant is also provided. The release device comprises a body having a proximal end which faces a user during use, and a distal end which is remote from the user during use, wherein an actuator is provided between the proximal end and the distal end. The release device is also used to release a medical implant from an insertion device, in the case of which the implant can be released by way of a relative motion between a first and a second insertion element. The method comprises at least the following steps: placing the actuator on the distal end of the body; actuating the actuator by applying a hydraulic pressure to a distal end of the body, thereby releasing the insertion element on the distal end of the insertion device by moving the insertion element in the direction of a proximal end of the insertion device; attaching the implant to the exposed insertion element; actuate the actuator by applying a hydraulic pressure at the proximal end of the body, thereby covering the implant with the insertion element which is slid in the direction of the distal end of the insertion device.

By way of the embodiment according to the invention, a method can be achieved which enables a release device to be manufactured in a manner that is user friendly, reliable and rapid. In this context, the term “actuator” also implies the circumstances of displacement of the actuator. In the case of the release of the insertion element—in particular the inner shaft—at the distal end of the insertion device, the actuation comprises displacement of the actuator in the direction of the proximal end of the body. Furthermore, in the case of the implant being covered by the insertion element—in particular the outer shaft—which is displaced in the direction of the distal end, actuation also implies displacement of the actuator in the direction of the distal end of the body. The expression “application of a hydraulic pressure” is intended to mean, in this case, that a pressure below atmospheric pressure is applied, such as 2 or 6 bar. This application of pressure can take place in a stepped manner or continuously.

In addition, a method for operating a release device is provided. The release device comprises a body having a proximal end which faces a user during use, and a distal end which is remote from the user during use, wherein an actuator is provided on the distal end, between the proximal end and the distal end. Furthermore, the release device serves to release a medical implant from an insertion device in the case of which the implant is disposed between a first and a second insertion element, wherein, by actuating the actuator via the application of a hydraulic pressure at the distal end of the body, the implant is released by way of a relative motion between the first and the second insertion element.

By way of the embodiment according to the invention, a method can be achieved which enables an implant to be released in a manner that is user friendly, reliable and rapid. In this case as well, the term “actuate” implies displacement and, in the case of the release of the implant, a displacement of the actuator in the direction of the proximal end of the body.

### DESCRIPTION OF THE DRAWINGS

The invention is explained in the following, as an example, in greater detail with reference to embodiments that are depicted in drawings. They show, in a diagrammatic representation:

FIG. 1 a section through a favorable embodiment of an insertion device and a release device;

FIG. 2 a detailed view of the release device from FIG. 1; and

FIG. 3 a detailed view of an alternative release device.

### DETAILED DESCRIPTION

Elements that are functionally identical or similar-acting are labelled using the same reference numerals in the figures. The figures are schematic depictions of the invention. They do not depict specific parameters of the invention. Furthermore, the figures merely show typical embodiments of the invention and should not limit the invention to the embodiments shown.

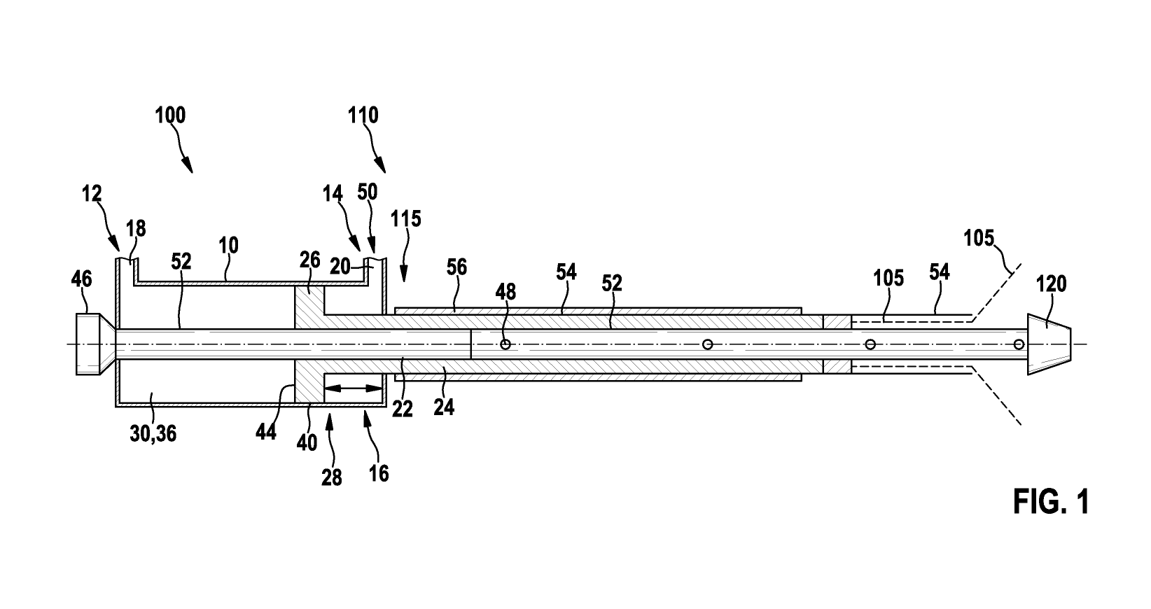

FIG. 1 shows a schematic side view of a favorable embodiment of a release device 100 of an insertion device 110 according to the invention, including an exposed housing 36 which forms a handle of the insertion device 110.

The insertion device 110 is, for example, a catheter comprising a shaft region 50 having two coaxially disposed insertion elements 52, 54, e.g. an inner shaft (insertion element 52) and, enclosing it, an outer shaft (insertion element 54) which can be enclosed by an outer sleeve 56. During use, i.e. during manufacture of the release device 100 or attachment of the implant 105 to the release device 100, or during implantation, the insertion device 110 faces a user by way of the proximal end 115 thereof. The implant 105 is placed on the distal end 120 of the shaft region 50 between the inner shaft and the outer shaft, and is intended for release at the implantation site in the animal body or human body.

The release device 100 serves to release the medical implant 105 from the insertion device 110. The implant 105 is disposed at an end 120 of the shaft region 50 opposite the housing 36, e.g. in the vicinity of a catheter tip. The implant 105 is placed around the inner insertion element 52, for example, and is released by way of a relative motion between the first and the second insertion elements 52, 54, as indicated in part in FIG. 1. To this end, the implant 105 is designed to be self-expanding.

The release device 100 comprises a body 10 having a proximal end 12 which faces the user during use, and a distal end 14 which is remote from the user during use. An actuator 16 is provided between the proximal end and the distal end 12, 14. The actuator 16 can be driven hydraulically and serves to generate a targeted relative motion between the first and the second insertion elements 52, 54 of the insertion device 110. In addition, the actuator 16 comprises a plunger rod 24 on the proximal end 28 of which a plunger 26 is disposed or attached. The plunger rod 24 and the plunger 26 are designed as one piece with the outer insertion element 54. Surfaces 40 of the plunger 26 extending parallel to the insertion element 54 form sealing contact surfaces with the body 10 which therefore forms a hydraulic cylinder 30 for the plunger 26. The actuator 16 or the plunger rod 24 and the plunger 26 comprise a passage 22 for axial guidance on the inner insertion element 52.

Furthermore, the body 10 comprises a valve 18 on the proximal end 12 thereof, and a valve 20 on the distal end 14 thereof. Both valves 18, 20 are designed as a luer lock for connection to a syringe 42. Using the syringe 42, a hydraulic fluid such as water can be introduced into the body 10 by way of each of the valves 18, 20, to move the actuator 16. If the hydraulic fluid comes in contact with an effective surface 44 of the plunger 26, the result is that the plunger rod 24 glides across the insertion element 52, such as the inner shaft of the catheter. As a result, the insertion element 54 or the outer shaft is also displaced in the direction of force. A speed of the motion of the plunger 26 and, therefore, the relative motion between the first and the second insertion elements 52, 54 of the insertion device 110 can be specified by way of a hydraulic pressure p1, p2. In addition, the speed of the relative motion can be monitored and/or regulated using a syringe manometer 38.

The release device 100 can be operated using at least two pressure ranges p1, p2. In this particular case, a first, low pressure range p₁, p₂ is provided for a slow relative motion between the first and the second insertion elements 52, 54, and therefore serves to slowly release the implant 105. A second, high pressure range p₁, p₂ is provided for a faster relative motion and is used for rapid release. The relative motion can take place rapidly or slowly by way of the pressure p₁, p₂ applied to the actuator 16 via the syringe 42 or the hydraulic fluid.

The length of each body 10 or the motional play of the plunger 26 is advantageously dimensioned such that it is at least as long as the length of the implant 105 to be released. In the case of a catheter as the insertion device 110 having a stent as the implant 105, the stent can be released in practical application starting with a slow speed up to a certain length, and can therefore be positioned very precisely. The stent can then be released completely at a higher speed. The slow release is particularly suitable for the start of the implant release at the implantation site.

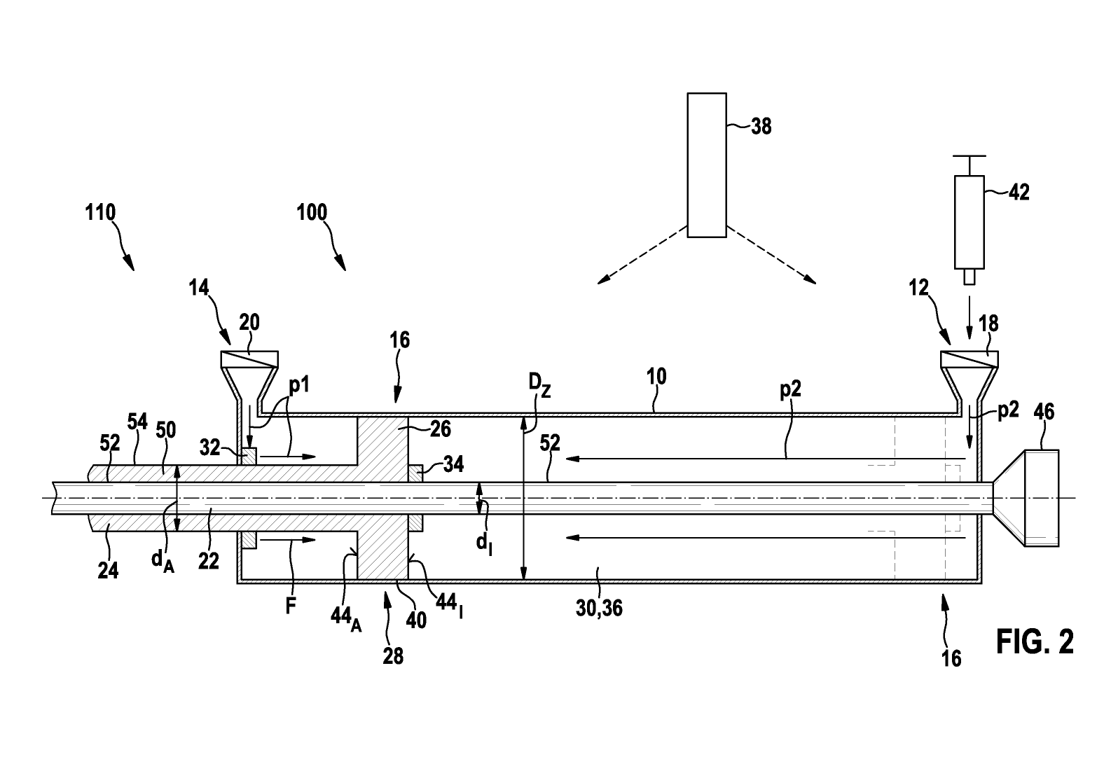

The body 10 or the housing 36 is transparent to permit monitoring of the movement of the plunger 26. The release device 100 has a seal 32 (see FIG. 2) for sealing an inner space of the body 10 with respect to the surroundings of the body 10. The seal 32 is disposed in the inner space between the body 10 and the insertion element 54, i.e. the outer shaft or the plunger rod 24, in the circumferential direction about the plunger rod 24. A further seal 34 is provided between the actuator 16 and the insertion element 52, i.e. the inner shaft, being disposed in the circumferential direction thereabout. The lumen of the insertion element 52 (inner shaft) can be ventilated and/or rinsed by way of a ventilation valve 46 (also known as a luer lock). Moreover, a plurality of ventilation holes 46 are provided between the outer shaft and the inner shaft.

A method for manufacturing the release device 100 including the implant 105 is described in the following with reference to FIG. 2. In a first step, the actuator 16 is placed on the distal end 14 of the body 10. This can be a position that has been preset by the manufacturer of the implant-free release device 100. In a second step, the actuator 16 is actuated by the application of a hydraulic pressure p1 at the distal end 14 of the body 10. This takes place by injecting the hydraulic fluid into the body 10 by way of the valve 20 using the syringe 42. This results in placement of the actuator 16 at the proximal end 12 of the body 10 (depicted using dashed lines). Due to the resulting motion of the insertion element 54 in the direction of a proximal end 115 of the insertion device 110, the insertion element 52 is released at the distal end 120 of the insertion device 110. Optionally, the parts of the body 10 that were filled with hydraulic fluid can be evacuated by way of the valve 20 before filling. It would also be possible, however, for the valve 20 to comprise a gas outlet which permits air located in the body 10 to escape in the direction opposite that of filling. To enable the actuator 16 to move more easily, the valve 18 is preferably opened at the proximal end 12 to allow air to escape.

In the subsequent, third step, the implant 105 is fastened onto the released insertion element 52, preferably by way of crimping. To this end, the pressure p1 on the syringe 42 is relieved by way of a 4-way tap of a tap bank (not shown). In a fourth and final step, the actuator 16 is re-actuated by applying a hydraulic pressure p2 at the proximal end 12 of the body 10 by injecting the hydraulic fluid into the body 10 by way of the valve 18 using the syringe 42. The actuator 16 is now placed at the distal end 14 of the body 10 once more. Furthermore, the implant 105 is covered by the insertion element 54 which is slid in the direction of the distal end 120 of the insertion device 110. The release device 100 or the insertion device 110 is now ready for implantation.

A method for operating the release device 100 or the insertion device 110 is carried out, after insertion of the insertion device 110 into the human body, by actuating the actuator 16, which is disposed at the distal end 14, by applying a hydraulic pressure p₁ at the distal end 14 of the body 10 once more by injecting the hydraulic fluid through the valve 20. As a result, the actuator 16 and the outer shaft are displaced, once more, to the proximal end 12 or in the direction of the proximal end 115 of the insertion device 110, and the implant 105 is released by way of the relative motion between the first and the second insertion elements 52, 54. The speed resulting from the motion of the colored (e.g. black) plunger 26 in the transparent cylinder 30 is monitored and regulated by way of the syringe manometer 38. The cylinder 30 can be marked using a legible scale, such as a mL scale, which is known to a person skilled in the art. The release in the body can be monitored using an xray procedure.

As mentioned above, the pressure p1, p2 can be varied as necessary, e.g. a pressure of 2 bar can be applied for a slow release, and a pressure of 6 bar can be applied for a faster release.

If the components of the release device 100 have the dimensions listed in the following tables as examples, then, according to TABLE 1, if a pressure p₁ of 6 bar, for example, is applied to the plunger 26 by way of the valve 20, then a force F of 39.58 N is applied to move the plunger 26 in the direction of the proximal end 12. According to TABLE 2, if a pressure p₂ of 5 bar is applied to plunger 26 by way of the valve 18, then a force F of 37.7 N is applied to move the plunger 26 in the direction of the distal end 14. In this case, D_{Z} represents a diameter of the cylinder 30, Dₖ is a diameter of the plunger 26, d_{A} is a diameter of the plunger rod 24 or the outer shaft (insertion element 54) and d_{I} is a diameter of the insertion element 52 or the inner shaft (see FIG. 2). The surface areas of the plunger rod 24, the insertion element 52 and the plunger 26 are calculated as πr2. In the case of p1, the effective surface area 44_{A} of the plunger 26 is determined by subtracting the surface area of the plunger rod 24 from the surface area of the plunger 26. In the case of p2, the effective surface area 44_{I} of the plunger 26 is determined by subtracting the surface area of the insertion element 52 from the surface area of the plunger 26.

TABLE 1

| | | |
|---|---|---|
| Cylinder 30 diameter D_{Z} | mm | 10.20 |
| Plunger 26 diameter D_{K} | mm | 10.00 |
| Plunger rod 24 diameter dA | mm | 4.00 |
| Surface area of the plunger rod 24 | mm² | 12.57 |
| Surface area of the plunger 26 | mm² | 78.54 |
| Effective surface area 44A | mm² | 65.97 |
| 1 bar* | N/cm² | 10.00 |
| Water injected with 6 bar pressure p1 | N/cm² | 60.00 |
| Force F on the plunger 26 | N | 39.58 |
| Length of the cylinder 30 | mm | 100.00 |
| Volume of the cylinder 30 | mm³ | 8171.28 |
| Volume of the cylinder 30 ml** | ml | 8.17 |

*1 bar = 10 N/cm²
**1 ml = 1 cm² = 1000 mm³

TABLE 2

| | | |
|---|---|---|
| Cylinder 30 diameter D_{Z} | mm | 10.20 |
| Plunger 26 diameter D_{K} | mm | 10.00 |
| Insertion element 52 diameter dI | mm | 2.00 |
| Surface area of the insertion element 52 | mm² | 3.14 |
| Surface area of the plunger 26 | mm² | 78.54 |
| Effective surface area 44I | mm² | 75.40 |
| 1 bar* | N/cm² | 10.00 |
| Water injected with 5 bar pressure p2 | N/cm² | 50.00 |
| Force F on the plunger 26 | N | 37.70 |
| Length of the cylinder 30 | mm | 100.00 |
| Volume of the cylinder 30 | mm³ | 8171.28 |
| Volume of the cylinder 30 ml** | ml | 8.17 |

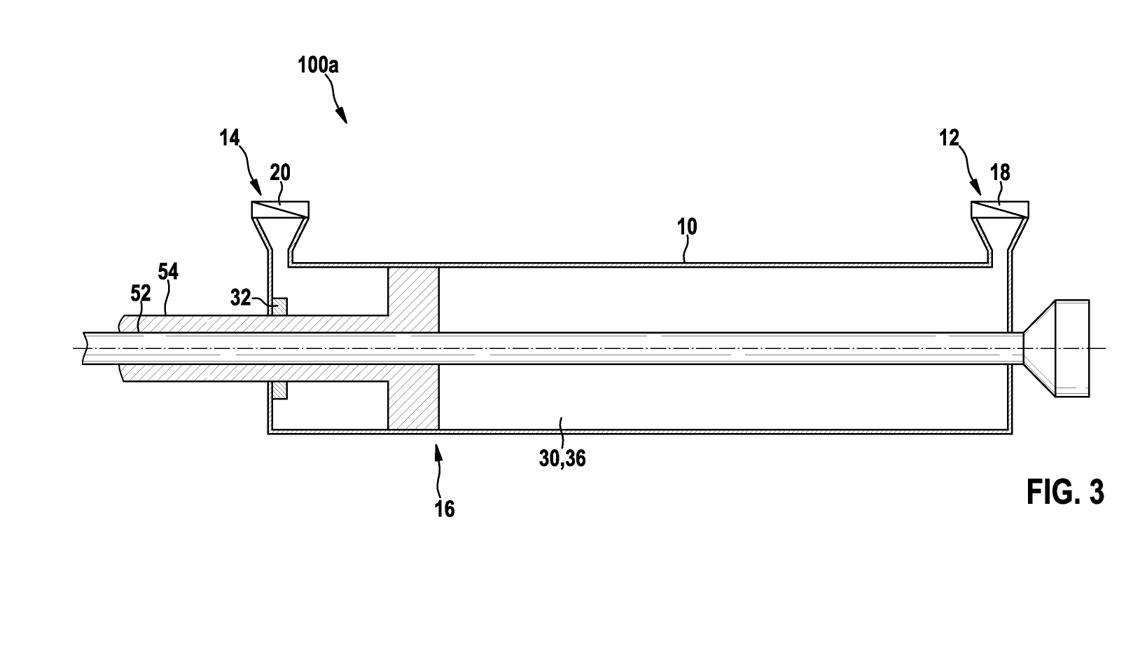

FIG. 3 shows an alternative release device 100a. It differs from the embodiment depicted in FIG. 2 in that the seal between the actuator and the inner shaft is omitted. This is possible because, in this case, the implant 105 was already attached in the insertion device 110 by the manufacturer of the release device 100a, and therefore the step of installing the implant 105 on the insertion device 110 is eliminated for the user of the release device 100a.

It will be apparent to those skilled in the art that numerous modifications and variations of the described examples and embodiments are possible in light of the above teaching. The disclosed examples and embodiments are presented for purposes of illustration only. Other alternate embodiments may include some or all of the features disclosed herein. Therefore, it is the intent to cover all such modifications and alternate embodiments as may come within the true scope of this invention.

What is claimed is:

1. A release device for releasing a medical implant from an insertion device, in the case of which the implant can be released by way of a relative motion between a first and a second insertion element, comprising a body having a proximal end which faces a user during use, and a distal end which is remote from the user during use, wherein an actuator is provided between the proximal and the distal end, wherein the actuator can be driven hydraulically to generate a targeted relative motion between the first and the second insertion element of the insertion device.

2. The release device according to claim 1, wherein the body comprises a valve at least on the distal end or one valve each on the proximal end and on the distal end.

3. The release device according to claim 2, wherein a hydraulic fluid for moving the actuator can be introduced into the body by way of the valve.

4. The release device according to claim 1, wherein a speed of the relative motion between the first and the second insertion elements of the insertion device can be specified by way of a hydraulic pressure (p1, p2).

5. The release device according to claim 4, wherein at least two pressure ranges (p1, p2) are provided, wherein a first, low pressure range (p1, p2) is provided for a slow relative motion, and a second, high pressure range (p1, p2) is provided for a faster relative motion.

6. The release device according to claim 1, wherein the actuator comprises a passage for the inner insertion element.

7. The release device according to claim 1, wherein the actuator comprises a plunger rod and a plunger, wherein the plunger is disposed on a proximal end of the plunger rod.

8. The release device according to claim 7, wherein the body comprises a hydraulic cylinder for the plunger.

9. The release device according to claim 7, wherein the plunger rod and/or the plunger are designed as one piece with the outer insertion element.

10. The release device according to claim 1, wherein a seal is provided, which is disposed between at least one insertion element and the body and/or the actuator.

11. The release device according to claim 1, wherein the body is in the form of a housing which is transparent in at least one region.

12. The release device according to claim 1, wherein a speed of the relative motion can be monitored visually.

13. The release device according to claim 1, wherein a speed of the relative motion can be monitored and/or regulated by way of a syringe manometer.

14. An insertion device for the insertion of a medical implant which can be released by way of a relative motion between a first and a second insertion element, comprising a release device for releasing the medical implant, comprising a body having a proximal end which faces a user during use, and a distal end which is remote from the user during use, wherein an actuator is provided between the proximal and the distal end, wherein the actuator can be driven hydraulically to generate a targeted relative motion between the first and the second insertion element of the insertion device.

15. A method for manufacturing a release device comprising an implant, comprising a body having a proximal end which faces a user during use, and a distal end which is remote from the user during use, wherein an actuator is provided between the proximal and the distal end and for releasing a medical implant from an insertion device, in which the implant can be released by way of a relative motion between a first and a second insertion element comprising at least the following steps:
placing the actuator at the distal end of the body;
actuating the actuator by applying a hydraulic pressure (p1) at the distal end of the body and therefore releasing the insertion element at a distal end of the insertion device by moving the insertion element in the direction of a proximal end of the insertion device;
attaching the implant to the exposed insertion element; and
actuating the actuator by applying a hydraulic pressure (p2) at the proximal end of the body, thereby covering the implant with the insertion element which is slid in the direction of the distal end of the insertion device.

16. A method for operating a release device, comprising a body having a proximal end which faces a user during use, and a distal end which is remote from the user during use, wherein, between the proximal and the distal end, an actuator is disposed at the distal end and for releasing a medical implant from an insertion device, in which the implant is disposed between a first and a second insertion element, wherein, by actuating the actuator via the application of a hydraulic pressure (p1) at the distal end of the body, the implant is released by way of a relative motion between the first and the second insertion element.
